(19) United States
(12) Patent Application Publication
(10) Pub. No.: US 2026/0061096 A1
(43) Pub. Date: Mar. 5, 2026

(54) Birefringent Intracorneal Lenticle and Methods of Use

(71) Applicant: Gebauer-Klopotek Patent Verwaltungs-UG, Neuhausen (DE)

(72) Inventor: Peter J. KLOPOTEK, Neuhausen (DE)

(21) Appl. No.: 19/314,282

(22) Filed: Aug. 29, 2025

Related U.S. Application Data
(60) Provisional application No. 63/689,806, filed on Sep. 2, 2024.

(51) Int. Cl. A61L 27/36; A61F 2/14

(52) U.S. Cl. CPC A61L 27/3604; A61F 2/1451; A61F 2/147; A61L 27/3691; A61F 2250/0053; A61L 2430/16

(57) ABSTRACT

In one aspect, a collagenous lenticule for use in intrastromal implantation is disclosed, which includes a decellularized lenticular body derived from a corneal donor source having an anterior surface and a posterior surface, where the decellularized lenticular body includes collagen fibers distributed therein such that the lenticular body exhibits birefringence to visible light and further exhibits sufficient optical clarity for intrastromal implantation. A lenticule according to the present teachings can be implanted in a patient's stromal layer to correct a refractive error of the eye, such as presbyopia.

### RELATED APPLICATION

The present application claims priority to Provisional Application No. 63/689,806 titled “Birefringent Intracorneal Lenticule and Methods of Use,” filed on Sep. 2, 2024, which is herein incorporated by reference in its entirety.

### TECHNICAL BACKGROUND

The present disclosure is generally directed to a birefringent intracorneal lenticule formed of decellularized donor corneal tissue as well as methods of using the lenticule for correcting refractive errors of a subject's eye.

### BACKGROUND

Intracorneal implants can be used to correct vision disorders, including refractive errors of the eye or otherwise induce refractive changes. Vision errors can pose significant problems for patients. Though many such vision disorders can be corrected using subtractive laser surgery, such techniques may lead to unwanted side effects. Recently, additive techniques have been developed that involve the transplantation of a lenticule into a patient's cornea after a flap has been cut and folded back to expose an intrastromal bed of the cornea. In such additive techniques, the shape of the implanted lenticule modifies the optical power of the patient's cornea by changing its curvature.

Conventional additive techniques, however, suffer from a number of shortcomings. For example, the availability of donor human corneas for forming lenticules is quite limited. Typically, conventional lenticules are implanted in a patient's cornea by forming a flap to expose a stromal bed followed by placing the lenticule on the exposed stromal bed and then covering the lenticule with the flap. The use of a flap may lead to weakening of the cornea leading to potential long term drift in refraction of the eye, as well as syndrome of dry eye or dangerous, albeit seldom, corneal ectasia. Techniques that utilize femtosecond laser radiation to make surgical cuts in the cornea for forming a corneal pocket into which a lenticule can be inserted are also known. But conventional lenticules are generally very pliable, which renders their insertion into such corneal pockets and the required spatial alignment with patient's visual axis, difficult.

The lenticules that cause shape transfer of anterior corneal surface for correcting refractive errors of the eye (or alternatively for inducing refractive changes) suffer also from several shortcomings. For example, in some cases, epithelial healing can diminish the shape transfer provided by the lenticule. In fact, conventional shape-transfer, small diameter lenticules (e.g., lenticules with diameters of less than 2.5 mm) lose most of their maximal refractive effect (located at the optical axis) to such epithelial “smoothing” process. Thus, it is difficult to predict the refractive performance of such conventional small diameter lenticules. Further, such conventional presbyopic lenticules can induce, e.g., due to flap thickness and/or epithelial smoothing, an optically multi-focal corneal shape. Further, shape transfer lenticules require precise alignment with the visual axis of the recipient's eye. Even a small misalignment can degrade the recipient's distant vision, especially in good lighting/illumination conditions. The visual axis of a patient's eye is, however, difficult to locate/track when a flap is lifted to place a lenticule in the stromal layer. Even experienced surgeons can miss the correct alignment of the lenticule by as much as 300 microns when implanting a shape transfer presbyopic lenticule in the cornea.

### SUMMARY

In one aspect, a collagenous lenticule for use in intrastromal implantation is disclosed, which includes a decellularized lenticular body derived from a corneal donor source having an anterior surface and a posterior surface. The decellularized lenticular body includes collagen fibers distributed therein such that the lenticular body exhibits birefringence to visible light and further exhibits sufficient optical clarity for intrastromal implantation.

In various embodiments, the birefringence of the lenticular body is characterized by a difference in a range of about 0.005 to about 0.1 for a refractive index of the lenticular body for visible light along at least two different spatial orientations within the lenticular body.

In various embodiments, the lenticular body exhibits a greater refractive index for visible light along an optical axis thereof relative to at least one axis perpendicular to the optical axis.

In various embodiments, the at least two orientations include a perpendicular and a tangential orientation, which is orthogonal to the perpendicular orientation. By way of example, the perpendicular orientation can be along an axis that is perpendicular to a putative plane that is tangential to a point on the anterior or the posterior surface of the lenticular body and the tangential orientation can be along (parallel to) one or more axes within the putative tangential plane. By way of example, the perpendicular orientation can be along an optical axis of the lenticular body and the tangential orientation can be along a direction that is orthogonal to the perpendicular axis. In various embodiments, the optical axis of the lenticular body coincides approximately with an axis of symmetry of the donor's cornea. In various embodiments, the optical axis of the lenticular body is along a visual axis of a recipient eye receiving the lenticule.

In various embodiments, the perpendicular orientation extends along an axis that is orthogonal to a putative surface that is tangential to any point on the anterior or the posterior surface and passes through that point and the tangential orientation extends along an axis within the putative surface that passes through that point.

In various embodiments, the tangential orientation is selected from among axes within the putative plane that pass through the point at which the putative tangential plane contacts the lenticule's anterior or posterior surface so as to maximize a difference in the refractive index of the lenticular body between the perpendicular and the tangential orientations.

In various embodiments, the perpendicular orientation extends along an axis that is orthogonal to the stromal layer and generally perpendicular to both the anterior and posterior sides of the stromal layer. The tangential orientation refers to any axis within a putative tangential plane at any given point on the corneal anterior surface, where the tangential plane refers to a mathematical plane that is both perpendicular to the local surface normal of the cornea at that given point and passes through that point, such that the tangential plane and the corneal surface share a single point of contact and no other points of intersection. The orientation of the tangential plane is determined by the first derivatives (slopes) of a mathematical function describing the corneal surface at that point.

In various embodiments, including those discussed above and further below, the refractive index of the lenticular body along the perpendicular orientation is greater than an average refractive index of human stromal layer by a value in a range of about 0.01 to about 0.07, e.g., in a range of about 0.02 to about 0.05, wherein optionally the perpendicular orientation is along an optical axis of the lenticular body.

By way of example, and without limitation, the refractive index of the lenticular body along the perpendicular orientation can be greater than 1.43 by a value in a range of about 0.01 to about 0.07, e.g., in a range of about 0.02 to about 0.05.

In various embodiments, the refractive index of the lenticular body along the tangential orientation is greater than 1.38, wherein optionally said tangential orientation is orthogonal to an optical axis of the lenticular body.

In various embodiments, the lenticular body has a maximum lateral dimension, e.g., a maximum diameter, of about 12 mm. By way of example, and without limitation, the lenticular body can have a lateral dimension, e.g., a diameter, in a range of about 0.5 mm to about 12 mm, e.g., in a range of about 0.5 mm to about 2.5 mm, or in a range of about 2.5 mm to about 5.5 mm, or in a range of about 3.5 mm to about 6.5 mm. By way of example, and without limitation, the lenticular body can have a substantially disk-like shape with a diameter in a range of about 0.5 mm to about 12 mm, e.g., in a range of about 0.5 mm to about 2.5 mm, or in a range of about 2.5 mm to about 5.5 mm, or in a range of about 3.5 mm to about 6.5 mm.

In various embodiments, the lenticular body can have a thickness less than about 100 micrometers (microns), and optionally in a range of about 10 microns to about 50 microns, or in a range of about 20 microns to about 40 microns.

In various embodiments, a concentration of the collagen fibers within the lenticular body is at least 20%, e.g., at least 30%, or at least 40%, or at least 50%, or at least 60%, or in a range from about 30% to about 70%.

In various embodiments, the collagen fibers are at least partially crosslinked, that is, at least some of the collagen fibers, regardless of their spatial locations within the lenticule, are crosslinked. In various embodiments, the collagen fibers within the lenticular body can be crosslinked within each triple-winding coil. Further, the collagen fibers in different lamellae can be crosslinked.

In various embodiments, the collagen fibers within the lenticular body substantially retain the layered arrangement of the collagen fibers of the donor cornea.

As noted above, in various embodiments, the lenticular body is decellularized. In various embodiments, any remaining cellular material in the decellularized lenticular body, as measured by residual RNA content, is less than one percent, or less than 0.1 percent, or less than 0.01 percent by weight of the original RNA content.

Further, in various embodiments, a lenticule according to the present teachings exhibits low immunoreactivity due to removal or degradation of immunogenic epitopes.

The lenticules according to the present teachings exhibit sufficient optical clarity to be suitable for intracorneal implantation, and more specifically for intra-stromal implantation. In some cases, the optical clarity of a lenticule according to the present teachings can be characterized by the degree of scattering exhibited by photons (and particularly visible photons) passing through the lenticule. By way of example, the lenticule can exhibit a scattering angle that is less than 4 arcminutes, or less than 2 arcminutes, or less than 1 arcminute.

In some cases, the optical clarity of a lenticule without a Fresnel or a diffractive pattern according to the present teachings can be characterized based on the probability of photons passing through the lenticule undergoing scattering events. By way of example, such a lenticule can exhibit an optical clarity that is characterized by at least 90 percent of visible photons passing through the lenticule with no scattering events. In various embodiments, the lenticule without a Fresnel or a diffractive pattern can exhibit an optical clarity that is characterized by a maximum scattering angle of visible photons passing through the lenticule being approximately equal to an angle corresponding to a diffraction limited resolution with an apodization of about 3 mm exhibited by the lenticule in the visible portion of the electromagnetic spectrum.

In various embodiments, at least one of the anterior and posterior surfaces of the lenticular body exhibits a curved profile. By way of example, both the anterior and the posterior surfaces of the lenticule can have a curved profile. In some such embodiments, the center of curvature of the anterior surface coincides with the center of curvature of the posterior surface. By way of example, the curved anterior and/or posterior surface can have a substantially hemispherical profile. The radius of curvature of any of the curved anterior and posterior surface can be, for example, in a range of about 3 mm to about 9 mm.

In various embodiments, at least one of the anterior and the posterior surface can include a diffractive or a Fresnel structure. By way of example, the Fresnel structure can function as a mono-focal refractive lens. In some embodiments, a Fresnel structure in which at least two different zones provide different values of refractive power, or a diffractive structure, may be used to allow the lenticule to function as a multifocal lens.

In a related aspect, a collagenous lenticule for use in intrastromal implantation is disclosed, which includes a decellularized lenticular body derived from a corneal donor source having an anterior surface and a posterior surface. The decellularized lenticular body includes collagen fibers distributed anisotropically within said lenticular body, thereby resulting in the lenticular body exhibiting different refractive indices for visible light along at least two spatial directions through the lenticular body. In various embodiments of such a collagenous lenticule, a difference between said different refractive indices is in a range of about 0.005 to about 0.1. In various embodiments, the collagenous lenticule exhibits a greater refractive index along its optical axis relative to any axis in plane perpendicular to the optical axis.

In a related aspect, a method for correcting a refractive error of a subject's eye is disclosed, which includes implanting a birefringent collagenous lenticule in a stromal layer of the subject's eye so as to correct said refractive error, wherein said birefringence of the lenticule is characterized by a difference in a range of about 0.005 to about 0.1 for refractive index of the lenticle along at least two different orientations in the lenticule.

In various embodiments, the birefringent collagenous lenticule can be implanted in the stromal layer of a patient's eye at a depth of at least 0.1 mm below an outer surface of the cornea, e.g., 0.1 mm below the corneal epithelial layer. By way of example, and without limitation, the birefringent collagenous lenticule can be implanted in the stromal layer at a depth in a range of about 0.1 mm to about 0.4 mm below the outer surface of the cornea.

In various embodiments, a pocket can be formed in the stromal layer of the cornea at a predetermined depth relative to the outer corneal epithelial layer of a subject. A channel can be formed to extend from the anterior epithelial surface of the cornea to the pocket. In certain embodiments, the pocket and/or the channel are created by directing femtosecond laser radiation into the corneal tissue under controlled guidance to achieve the desired geometry and location of the channel and the pocket. The channel can be dimensioned to permit the insertion of a lenticule (e.g., in a folded form) therein, where the lenticule can be advanced through the channel to reach the pocket, thereby positioning the lenticule within the stromal layer in a manner that facilitates its stable implantation.

In various embodiments, the refractive index of the lenticule that is implanted in the patient's stromal layer can be equal to or greater than 1.48 along its optical axis. By way of example, such a lenticule can have a refractive index in a range of about 1.48 to about 1.5 along its optical axis.

Further, in various embodiments, the lenticule that is implanted in the patient's stromal layer can have a thickness in a range of about 10 microns to about 100 microns, e.g., in a range of about 20 microns to about 90 microns, or in a range of about 30 microns to about 80 microns, or in a range of about 40 microns to about 70 microns, or in a range of about 50 microns to about 60 microns.

A variety of refractive errors can be corrected via implantation of lenticules according to the present teachings in a patient's eye. Some examples of such refractive errors include, without limitation, presbyopia, high astigmatism, hyperopia, myopia, and irregular refractive disorder.

A lenticule according to various embodiments can have a thickness in a range of about 10 microns to about 100 microns. Such thin lenticules can achieve the correction of a refractive error of a patient's eye without shape transfer.

As noted above, one example of a refractive error that can be corrected via implantation of a lenticule according to the present teachings in the stromal layer of a patient's eye is presbyopia. It has been discovered that a lenticule according to embodiments of the present teachings having a diameter in a range of about 0.5 mm to about 2.5 mm is particularly suitable for the correction of presbyopia.

In various embodiments in which a lenticule according to the present teachings is utilized for correcting presbyopia, the lenticule can provide an optical power, for example, in a range of about 1 to about 6 Diopters.

In various embodiments in which a lenticule having a Fresnel lens is used for correcting a refractive error, the optical power of the lenticule can be, for example, in a range of about +/−1 to about +/−10 Diopters.

In a related aspect, a method of correcting a refractive error of a subject's eye is disclosed, which includes implanting a collagenous lenticule in a stromal layer of the subject's eye so as to correct said refractive error, wherein said lenticule exhibits a refractive index in a range of about 1.44 to about 1.5 and has a thickness in a range of about 10 microns to about 100 microns. In various embodiments of the method, the lenticule corrects the refractive error without causing shape transfer of an anterior surface of the cornea. By way of example, in some embodiments in which the refractive error is presbyopia, the lenticule has a diameter in a range of about 0.5 mm to about 2.5 mm.

In a related aspect, a collagenous lenticule for use in intrastromal implantation is disclosed, which includes a decellularized lenticular body derived from a corneal donor source having an anterior surface and a posterior surface, said decellularized lenticular body having collagen fibers distributed anisotropically therein, thereby resulting in the lenticular body exhibiting different refractive indices for visible light along at least two spatial directions through the lenticular body. In some embodiments, a difference between said different refractive indices is in a range of about 0.005 to about 0.1.

In a related aspect, a method of fabricating a collagenous lenticule is disclosed, which includes decellularizing a lenticular body derived from a corneal donor source, applying concurrently a compressive force and a stretching force to said decellularized lenticular body so as to cause a reduction in thickness of said lenticular body while causing a lateral expansion thereof, wherein said compressive force is along a direction substantially perpendicular to collagen fibrils lamellae of said lenticular body, and causing cross-linking of at least a portion of the collagen fibrils in said lenticular body, preferably while the lenticular body remains under compression.

In a related, a device for fabricating a collagenous lenticule is disclosed, which includes a pair of stamping components separated from one another by a gap in which a lenticular body derived from a corneal donor source can be placed, wherein at least one of said stamping components is movable along a direction substantially orthogonal to lamellar arrangement of collagen fibrils in said lenticular body. A compliant rubber-like element is coupled to said movable stamping component such that application of a compressive force to said lenticular body via movement of said movable stamping component results in a lateral deformation of said compliant rubber-like element resulting in application of a stretching force to said lenticular body.

In some embodiments, at least one of said stamping components allows transmission of UV radiation for causing crosslinking of collagen fibrils in said lenticular body.

Further understanding of various aspects of the present teachings can be obtained by reference to the following detailed description in conjunction with the associated drawings, which are described briefly below.

### BRIEF DESCRIPTION OF THE DRAWINGS

The drawings are provided for illustrative purposes only and are not necessarily to scale:

FIG. 1A is a schematic perspective view of a lenticule according to an embodiment of the present teachings,

FIG. 1B is a top view of the lenticule illustrated in FIG. 1A,

FIG. 1C is a cross-sectional side view of the lenticule illustrated in FIG. 1A,

FIG. 1D schematically depicts a lenticule according to an embodiment that includes a diffractive pattern on its anterior surface,

FIG. 1E shows, by way of comparison, a lens having the same diameter and optical power as the lenticule illustrated in FIG. 1D, but without the Fresnel pattern,

FIG. 2A schematically depicts the lenticule shown in FIG. 1A with a putative plane tangent to an apex of the anterior surface of the lenticule for defining two axes, where the refractive index of the lenticule along one axis is different than the respective refractive index along the other axis,

FIG. 2B schematically depicts the lenticule shown in FIG. 1A with a putative off-centered plane that is tangent to the anterior surface of the lenticule for defining two other axes, where the lenticule exhibits different refractive indices along those axes,

FIG. 3A schematically depicts two adjacent corneal lamellae,

FIG. 3B is a graph of a brightness distribution that can be utilized in determining the optical clarity of a lenticule,

FIG. 4A depicts a step in a method for implanting a lenticule into the stromal layer of a patient's cornea in which a corneal pocket is formed in the cornea using a plurality of femtosecond laser pulses,

FIG. 4B depicts the insertion of a lenticule according to an embodiment into the pocket formed in the cornea as shown in FIG. 4A,

FIG. 4C shows centering the lenticule implanted into the pocket formed in the cornea,

FIG. 5A schematically depicts an apparatus for fabricating a lenticule according to various embodiments,

FIG. 5B is an enlarged view of a section of the apparatus shown in FIG. 5A, more clearly illustrating a scaffold being processed by the apparatus,

FIG. 5C is a top schematic view of a scaffold being processed by the apparatus of FIG. 5A,

FIG. 6A is a perspective view of the apparatus illustrated in FIG. 5A,

FIG. 6B is a partial perspective view of the apparatus illustrated in FIG. 5A,

FIG. 7A is a schematic cross-sectional view of another apparatus for fabricating a birefringent lenticule according to various embodiments,

FIG. 7B schematically depicts simultaneous application of a compressive and a stretching force to a decellularized lenticular body using the apparatus shown in FIG. 7A, and

FIG. 7C schematically depicts decellularized lenticular body of FIG. 7B after having been subjected to compressive and stretching forces to achieve a desired thickness and diameter.

### DETAILED DESCRIPTION

This invention is not limited to the particular processes, compositions, or methodologies described, as these may vary. The terminology used in the description is for the purpose of describing the particular versions or embodiments only, and is not intended to limit the scope of the present invention. Unless defined otherwise, all technical and scientific terms used herein have the same meanings as commonly understood by one of ordinary skill in the art. All publications mentioned herein are incorporated by reference in their entirety.

The term “biological sample” refers to tissue, cells, cellular extract, homogenized tissue extract, or a mixture of one or more cellular products. The biological sample can be used or presented in a suitable physiologically acceptable carrier.

As used herein and in the appended claims, the singular forms “a,” “an,” and “the” include plural references unless the context clearly dictates otherwise. Thus, for example, reference to a “cell” is a reference to one or more cells and equivalents thereof known to those skilled in the art, and so forth.

As used herein, the term “about” means plus or minus 10% of the numerical value of the number with which it is being used. Therefore, about 100 μm means in the range of 90 μm-110 μm.

The terms “animal,” “patient,” or “subject” as used herein include, but is not limited to, humans and non-human vertebrates such as wild, domestic and farm animals. The terms “animal,” “patient,” or “subject” also refer to the recipient of a corneal lenticule transplant.

The term “xenograft” refers to tissue collected from animals for donation, including pigs (porcine), bovine, apes, monkeys, baboons, other primates, and any other vertebrates.

The term “allograft” refers to tissue collected from a donor species for donation to a member of the same species, e.g., corneal tissue obtained from a human donor to implantation in a human recipient.

Generally speaking, the term “tissue” refers to any aggregation of similarly specialized cells which are united in the performance of a particular function.

The term “lenticule” refers to a decellularized, processed donor corneal tissue ready for implantation into a recipient's stromal bed.

The terms “collagen concentration” and “collagen percentage” are used interchangeably herein and refer to amount of collagen present in a lenticule or scaffold. This concentration or percentage can be measured as the fractional weight of a completely desiccated lenticule, e.g., a vacuum desiccated lenticule, relative to the weight of the lenticule before desiccation (in full equilibrium with water). In some cases, ethanol can be used to improve desiccation.

The term “intracorneal” in the context of lenticule implantation refers to any procedure in which a lenticule is placed in or on the cornea. One type of intracorneal implantation is “intrastromal” implantation, a procedure in which a lenticule is placed within the stroma of the eye, without excision of the anterior Bowman's membrane or epithelium, e.g., by the folded back of a flap of anterior tissue or by direct insertion via a lateral approach. Other types of intracorneal use of lenticules include deep anterior lamellar keratoplasty (DALK) and penetrating keratoplasty (PK), in which a lenticule replaces an anterior segment of the eye completely. Yet another applicable “intracorneal” procedure is so-called “epikeratoplasty.”

The term “axial” refers to a direction relative to the orientation of the lenticule. Typically, the shaped lenticule will be curved in spheroidal or ellipsoidal disc-like shape and the axial direction or “axis” will be generally perpendicular to the center of the disc. Unless otherwise indicated, “axial” is also generally almost parallel to, or coaxial with, the visual or optical axis of an eye from which the tissue segment is excised or the recipient eye where the lenticule is designed to be implanted. (In a natural eye, the optical axis typically goes almost, through the center of the cornea and should not be confused with visual axis which may deviate from the optical axis by approximately 4 to 7 deg.).

The term “refractive index,” which is also known as index of refraction, is a dimensionless number that quantifies how much the speed of light is reduced in a medium compared to its speed in vacuum. It is defined mathematically as n=c/v, where n denotes the refractive index, c is the speed of light in vacuum, and v denotes the speed of light in the medium. The refractive index of a lenticule according to the present teachings can be measured as described in the Example section below.

The term “birefringence” refers to an optical property of a material whose index of refraction (called also refractive index) varies depending on the propagation direction of incident light as well as on polarization (the type of polarization and its orientation) of that incident light. This phenomenon can and often does arise from the material's anisotropy, i.e., at least one physical property of the material differs with direction within the material.

The term “optical axis,” as used herein refers to an imaginary straight line that passes through the geometrical center of an optical system. For a lenticule, the optical axis of the lenticule will be aligned with the visual axis of a recipient's eye. For lenticules that exhibit rotational symmetry, the optical axis coincides with the axis of symmetry. Further, any ray that is incident on an optical surface of a lenticule along the optical axis passes through and exits the lenticule without a change in its propagation direction.

The term “visible range of the electromagnetic spectrum” refers to radiation with a wavelength in a range of about 400 nm to about 700 nm.

A “refractive error” of the eye as used herein refers to a condition of the eye in which light fails to focus properly on the retina. This can occur due to abnormalities in the optical properties or geometry of the eye, including, but not limited to, variations in the shape and/or curvature of the cornea, alterations in pliability and/or shape of the crystalline lens, changes in the axial length (e.g., from the cornea to the retina) of the eyeball, and combinations of these factors or other anatomical abnormalities that can affect how light is focused onto the retina.

This disclosure relates to a decellularized corneal lenticule formed from allograft and/or xenograft sources and methods of forming a lenticule from donor corneal tissue as well as the use of such a lenticule for correcting a refractive error of a patient's eye. The disclosed decellularized corneal lenticules are intended to be used to correct abnormal refractive conditions, such as myopia, hyperopia, presbyopia, astigmatism, or irregular distortions. The lenticules according to various embodiments can also be used to correct presbyopia, which (dependent on the point of view) could be classified as “natural refractive error” and not as “abnormal refractive conditions”.

The present disclosure generally describes intracorneal lenticules, and particularly lenticules that can be implanted in the stromal layer of a patient's eye for correcting a refractive error of the eye. In various embodiments, the lenticules exhibit an optical anisotropy that is greater than the optical anisotropy exhibited by natural donor cornea from which the lenticule is fabricated so as to increase the refractive index of the lenticule along its optical axis at the expense of reducing the refractive index in directions orthogonal to the optical axis. In various embodiments, the increase in the refractive index along the optical axis allows forming small-diameter, thin lenticules that can advantageously be used to correct refractive errors of the eye without any shape transfer effects (i.e., without changing the curvature of the anterior surface of the recipient's eye to any appreciable extent, if at all).

As discussed in more detail below, in various embodiments, a lenticule according to the present teachings can exhibit a refractive index that is somewhat larger than the refractive index of a stromal layer into which the lenticule is implanted so as to correct the eye's refractive error. In various embodiments, a lenticule according to the present teachings corrects the refractive error via direct optical action of the lenticule (i.e., via the difference between the refractive index of the lenticule and that of the patient's stroma), rather than indirectly by changing the exterior curvature of the cornea. In fact, in many embodiments, a lenticule according to the present teachings is implanted deep in the stromal layer, where its effect on the curvature of the corneal anterior surface will be minimal, if at all present.

In various embodiments, due to a modest difference between the refractive index of a lenticule and that of a recipient's stroma, the lenticules have a small diameter, for example, to be used as presbyopic lenticules. Further, in some embodiments, a diffractive pattern, such as a Fresnel pattern, can be applied to the lenticule surface allowing/enabling the construction of a monofocal lenticule that exhibits a modest difference in its refractive index relative to that of the stroma, but with a larger diameter, e.g., a diameter in a range of about 2 mm to about 6 mm. The refractive properties of such a lenticule having a Fresnel or a diffractive pattern can be used to correct a refractive error of the eye without any shape transfer effects.

Moreover, a lenticule according to the present teachings exhibits birefringence as characterized, e.g., by a difference of greater than 0.005 in the refractive index along at least two distinct spatial orientations within the lenticule (i.e., at least two non-parallel orientations). For example, one direction can be along the lenticule's optical axis and another direction can be in a plane that is orthogonal to the optical axis.

Crystalline birefringent materials exhibit optical anisotropy with respect to typically one to three axes. Further, birefringence is associated with the behavior of light with respect to its polarization and propagation direction within the birefringent material.

In various embodiments, the birefringence exhibited by a lenticule according to the present teachings is modest and the lenticule is sufficiently thin such that most polarization effects are of no importance for the vision of a patient receiving the lenticule. Further, unlike many crystalline birefringent materials, a lenticule according to various embodiments of the present teachings can exhibit, as a function of the location (and its angular details), a large multiplicity of optical axes but only one “privileged” axis that coincides with the lenticule's geometrical axis of symmetry.

As discussed in more detail below, in various embodiments, the birefringence of a lenticule according to the present teachings can be characterized as the difference between the refractive indices of the lenticule along a perpendicular and at least one tangential orientation, i.e., an orientation orthogonal to the perpendicular orientation. In various embodiments, the refractive index of the lenticule along its optical axis (e.g., corresponding to the lenticule's geometrical axis of symmetry) is the parameter that is of primary significance in imparting clinical utility to the lenticule. In various embodiments, a lenticule according to the present teachings is constructed to maximize its refractive index along the optical axis, which in turn leads to the lenticule exhibiting some degree of birefringence.

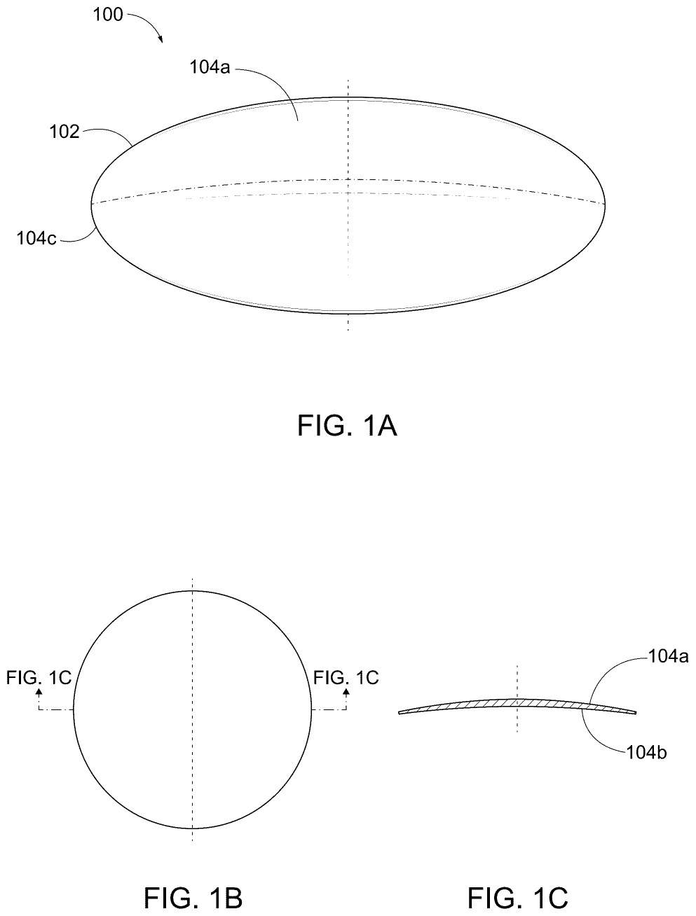

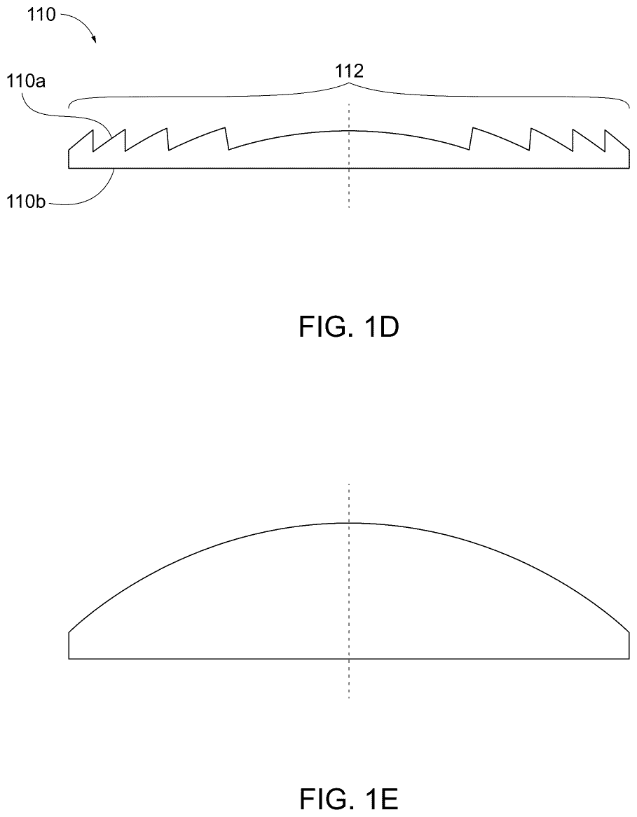

With reference to FIGS. 1A, 1B, and 1C, a birefringent lenticule 100 according to an embodiment, which is suitable for intrastromal implantation, e.g., to correct a refractive error of a patient's eye, includes a lenticular body 102 that extends between an anterior surface 104a and a posterior surface 104b with a lateral surface 104c between the anterior surface 104a and the posterior surface 104b. In this embodiment, both the anterior and the posterior surfaces have a convex hemispherical shape, albeit with different radii of curvature, though in other embodiments, one or both of the anterior and posterior surfaces may have other shapes, such as a flat or a concave profile. Further, in some embodiments the anterior and the posterior surfaces of the lenticular body can exhibit the same radius of curvature, or alternatively can be co-concentric (have difference of radii matching the lenticule thickness).

The birefringent lenticule 100 exhibits two different refractive indices along at least two different orientations (i.e., two orientations separated by a non-zero angle, that is, two orientations that are not parallel to one another). By way of example, the difference in the refractive index of the lenticular body along those different orientations can be in a range of about 0.005 to about 0.1, e.g., in a range of about 0.005 to about 0.1, or in a range of about 0.02 to about 0.03 or in a range of about 0.03 to about 0.04, or in a range of about 0.05 to about 0.06, or in a range of about 0.06 to about 0.07, or in a range of about 0.08 to about 0.09, or in a range of about 0.09 to about 0.1.

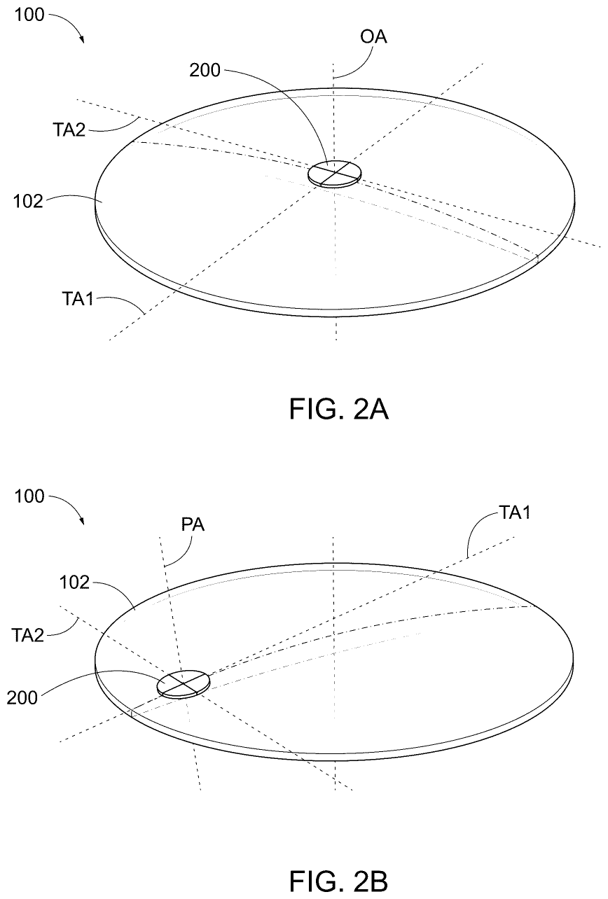

By way of illustration, FIG. 2A schematically depicts orientations along which the lenticular body exhibits different refractive indices. More specifically, FIG. 2A depicts the lenticule 100 shown in FIG. 1A with a putative plane 200 that is tangent to the lenticule at the apex (herein the center) of its convex anterior surface. In this embodiment, an optical axis (OA) of the lenticular body is perpendicular to the putative tangential plane 200 and passes through the point at which the tangential plane 200 contacts the anterior surface of the lenticule (herein referred to as the “contact point”). Although in this example the lenticular body is fully symmetric relative to the optical axis, in other cases, the lenticular body may exhibit a generally asymmetric shape relative to the optical axis.

In various embodiments, the refractive index of the lenticular body along a perpendicular orientation that extends along/parallel (e.g., coincident with) the optical axis is greater than the refractive index of the lenticular body along any tangential orientation that extends along/parallel (e.g., is coincident with) any of the axes within the putative tangential plane 200 that passes through the contact point. For example, the refractive index of the lenticule 100 along the optical axis can be different than the lenticule's refractive index along orientations that are parallel to tangential axes (TA1) and (TA2), or in fact, any other tangential axis within the putative tangential plane 200.

In various embodiments, the refractive index of the lenticular body along different tangential orientations and/or at different locations along a tangential orientation may be different, though less than the refractive index of the lenticule along its optical axis. Such variations, however, do not adversely affect the functioning of a lenticule according to the present teachings as the lenticules are thin and their refractive properties are dominated by their refractive index along the optical axis.

By way of example, the difference in the refractive indices of the lenticular body along the optical axis and a tangential orientation can be in a range of about 0.005 to about 0.1, including any of the sub-ranges identified above.

The characterization of the birefringence of a lenticule according to the present teachings is not limited to using a difference between the refractive index of the lenticular body along one orientation that is along/parallel the optical axis and another orientation that is along a tangential axis orthogonal to the optical axis. Rather, the difference in the refractive index of the lenticular body along off-centered perpendicular and tangential axes can also be utilized to characterize the birefringence of a lenticule according to various embodiments of the present teachings.

By way of illustration, FIG. 2B schematically depicts the lenticule 100 with a putative plane 202 that is tangent to the anterior surface of the lenticule 100 at an off-centered point that is closer to the lenticule's periphery than the point at which the optical axis contacts the lenticule's anterior surface. In this case, the tangential plane 202 is oriented such that its extension would cross the optical axis (OA). In other words, the local perpendicular axis PA (FIG. 2B) is not parallel to the optical axis (OA) (FIG. 2A).

With continued reference to FIG. 2B, an axis (PA) (which is herein also referred to as “a perpendicular axis”) is perpendicular to the tangential plane 202 and passes through the contact point between the tangential plane 202 and the anterior surface of the lenticule. In various embodiments, the refractive index of the lenticular body along an orientation that is along the perpendicular axis (PA) is different than the refractive index of the lenticular body along an orientation that is parallel to any axis within the tangential plane that passes through the contact point at which the putative tangential plane 202 contacts the anterior surface of the lenticule. In various embodiments, the refractive index of the lenticular body along the perpendicular axis (PA) does not exhibit any significant variation as a function of the location of the perpendicular axis (PA) on the lenticule's surface.

In various embodiments, to characterize the birefringence of the lenticule, at least one axis within the tangential plane (herein referred to also as a “preferred tangential axis”) is selected that maximizes the difference in the refractive index of the lenticular body between the perpendicular and tangential orientations. For example, in this embodiment, the tangential axis TA1 may be such a preferred tangential axis. By way of example, the difference in the refractive index of the lenticular body along an orientation along the perpendicular axis (PA) relative to an orientation parallel to TA1 can be in a range of about 0.005 to about 0.1, including any of the sub-ranges identified above.

Without being limited to any particular theory, in various embodiments, an anisotropy in the statistical distribution of the collagen fibers within the lenticular body and/or that of the glycosaminoglycans (GAGs) in the decellularized cross-linked corneal tissue forming the lenticular body 102 can give rise to the anisotropy in the refractive index of the lenticule in the visible range.

In particular, in various embodiments, a lenticule according to the present teachings exhibits an extra anisotropy relative to the natural donor cornea, where the extra anisotropy results in an increase in the refractive index of the lenticule along perpendicular axes (such as the optical axis or the perpendicular axis (PA) discussed above) at the expense of a decrease in the refractive index of the lenticular body along tangential directions (i.e., direction orthogonal to the perpendicular axes).

The cornea can be generally considered to include 5 layers, from anterior to posterior: the corneal epithelium, a thin but dense top stromal layer (typically referred to as Bowman's membrane in human eyes), the corneal stroma proper, Descemet's membrane, and corneal endothelium. The corneal epithelium is composed of about 6 layers of non-keratinized stratified squamous epithelium cells, which are fast growing and easily regenerated. The anterior stromal layer (e.g., Bowman's membrane) is a tough layer composed mostly of randomly organized, tightly woven collagen type I fibrils. The corneal stroma is a thick, transparent layer consisting of collagen type I fibers arranged in parallel layers. The Descemet's membrane is a thin acellular layer that serves as the basement membrane of the corneal endothelium and is composed of less rigid collagen type IV fibrils. Finally, the corneal endothelium is composed of simple squamous or low cuboidal monolayer of mitochondria-rich cells.

Stromal collagen fibrils (herein also referred to as collagen fibers) are long polymeric (polypeptide) strings. They are triple-winded proteins. The length of a single collagen fibril is nearly macroscopic, and so each of the fibrils individually can be a strong scatterer of light. The fact that stroma is transparent in the axial direction is the result of negative summation of all these strongly scattering contributions. The corneal collagen fibers are arranged in a plurality of lamellae, where the collagen fibers within each of the lamellae are substantially aligned. By way of example, FIG. 2C illustrates schematically two such lamellae 301 and 302 with a plurality of substantially aligned collagen fibrils 301a and 302a within those lamellae.

In some various embodiments, the refractive index of the lenticule along a tangential orientation can be smaller than a respective refractive index along an orientation that is generally perpendicular to the orientation of the collagen fibers.

The above examples illustrate that the anisotropy in the refractive index of the lenticular body can be characterized relative to different pairs of orientations (axes) within the lenticular body. In general, many such orientation pairs can be used for characterizing the refractive index anisotropy of the lenticular body.

In this embodiment, the lenticular body 102 is formed of decellularized corneal tissue in which at least a portion of collagen fibers are crosslinked to enhance mechanical stability of the lenticular body and inhibit its swelling once implanted in a subject's stromal layer, as discussed in more detail below.

Moreover, the lenticular body 102 exhibits optical clarity (herein also referred to as transparency) in the visible range of the electromagnetic spectrum such that it is suitable for implantation in the stromal layer of human cornea for correcting a refractive error, such as those discussed herein.

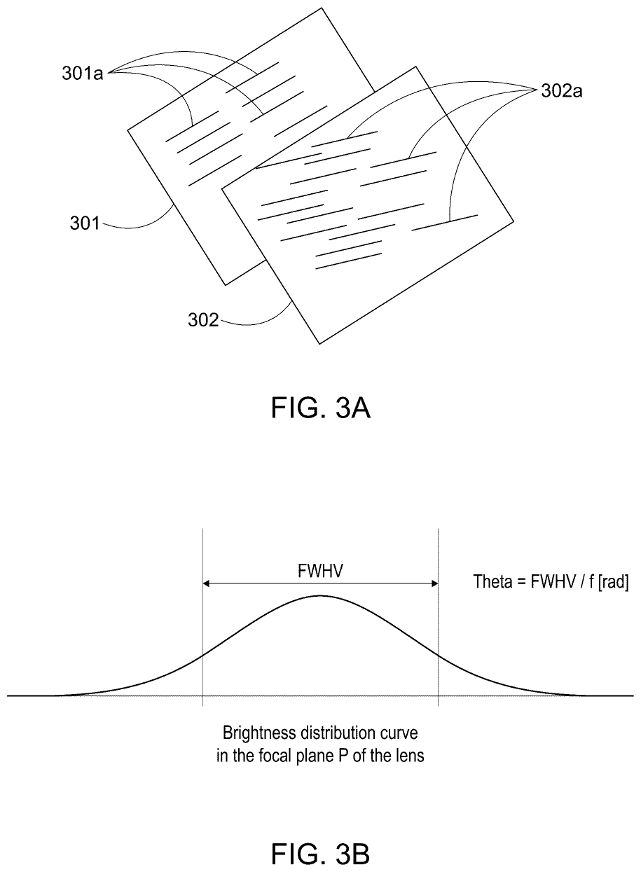

By way of example, FIG. 3B is an illustration of a method of quantifying transparency of a lenticule by measuring the lenticule's scattering angle (referred herein as “θ or “theta”). FIG. 3B illustrates a brightness distribution curve as measured in the focal plane of the lenticule. This Gaussian-like distribution represents the sum of all the contributions of scattered wave fronts. The more scattering in the lenticule, the larger is the width of the detected beam. One measure of scattering (the degradation of optical clarity) is the full-width, half vertical maximum (FWHV) as shown in FIG. 3B. It should be understood that the Gaussian-like curve of FIG. 3B is an idealization and real brightness curves can be distorted by noise or other spurious signals. Multiple measurements and averaging (or other known noise reducing signal processing techniques) can be used to obtain the best data representation of scattering by the lenticule. The FWHV value divided by the focal length, f, provides a measurement of the angular spread of the beam (in radians): θ=FWHV/f [rad].

Theta is dependent only on the amount of scattering and can also be expressed in terms of arcminutes using the conversion formula: 1 arcmin=291 microrad. Lenticules manufactured by the methods disclosed herein exhibit satisfactory transparency (e.g., for intracorneal implantation) if the angle θ is less than 4 arcminutes, preferably, less than 3 or 2 arcminutes, more preferably in some instances, less than 1 arcminute.

The optical clarity of a lenticule according to various embodiments of the present teachings can also be described in terms of the probability that a photon passing through the lenticule would undergo a scattering event. In various embodiments of lenticules without a Fresnel or diffractive pattern, at least 90%, or at least 95%, or at least 99% of visible photons passing through the lenticule undergo no scattering event. Such a low scattering cross section of the photons results in a low-angle scattering of the photons. By way of example, in various embodiments, the maximum scattering angle of visible photons passing through the lenticule can be approximately equal to an angle corresponding to a diffraction limited resolution exhibited by the apodised (to diameter of about 3 mm) lenticule in the visible portion of the electromagnetic spectrum. For example, such a diffraction-limited resolution angle (θ) can be defined as: θ=1.22λ/D, where λ denotes the wavelength of the radiation and D denotes the diameter of the apodised lenticule. In various embodiments, a presbyopic lenticule according to the present teachings has a diameter of less than 3 mm. As such, no apodization needs to be considered for such presbyopic lenticules.

Without being limited to any particular theory, the transparency of the lenticule can be maintained while fabricating the lenticule from donor corneal tissue by ensuring that the orientations of the collagen fibrils within various lamellae in the stromal tissue are preserved, i.e., the respective orientations of the collagen fibrils in the lenticule are substantially similar to those within natural stromal layer of the donor's corneal tissue. Again, without being limited to any particular theory, as the stretching applied to a scaffold harvested from a donor's corneal tissue is along the transverse tangential direction and the fibrils are also (mostly) radially oriented (as they are on the polar orbits), as long as the donor stromal tissue is harvested around the donor's eye axis of symmetry, the alignment will be preserved.

Referring again to FIGS. 1A and 1B, the refractive index of the lenticule 100 is greater than that of an average human stromal layer. By way of example, the refractive index of the lenticular body 102 of the lenticule 100 along the optical axis can be greater than 1.43. For example, the refractive index of the lenticular body 102 along the optical axis can be greater than 1.43 by a value in a range of about 0.01 to about 0.07, i.e., the refractive index of the lenticular body along the optical axis (OA) can be in a range of about 1.44 to about 1.5. Further, in various embodiments, the refractive index of the lenticular body 102 along the transverse axis (TA) can be greater than 1.37 by a value in a range of about 0.01 to about 0.1 i.e., the refractive index of the lenticular body along the TA can be in a range of about 1.38 to about 1.48.

With continued reference to FIGS. 1A, 1B and 1C, in this embodiment, the lenticular body 102 has a disk-like shape with a diameter (D) in a range of about 0.5 mm to about 12 mm, e.g., in a range of about 1 mm to about 10 mm, or in a range of about 2 mm to about 9 mm, or in a range of about 3 mm to about 8 mm, or in a range of about 4 mm to about 7 mm, or in a range of about 5 mm to about 6 mm. Further, the lenticular body 102 can have a maximum thickness in a range of about 10 microns to about 100 microns, e.g., in a range of about 20 microns to about 90 microns, or in a range of about 30 microns to about 80 microns, or in a range of about 40 microns to about 70 microns, or in a range of about 50 microns to about 60 microns. Lenticules according to various embodiments having a diameter in a range of about 0.5 to about 2.5 mm are particularly suitable for use in correcting presbyopia. In particular, such lenticules can correct a patient's presbyopia without adversely affecting the patient's distant vision.

In some embodiments, a Fresnel and/or a diffractive pattern can be superimposed on at least one of the anterior or posterior surfaces of a lenticule according to the present teachings. By way of example, FIG. 1D schematically depicts such a lenticule 110 that includes an anterior surface 110a and a posterior surface 110b. The anterior surface 110a includes a diffractive pattern 112 that functions as a mono-focal Fresnel lens when immersed in stromal tissue. In various embodiments, the use of such a Fresnel and/or diffractive pattern allows the lenticule to be thin (e.g., having a maximum thickness of less than 100 microns) with a larger diameter (e.g., a diameter between about 5.5 mm and about 6.5 mm) while providing the requisite optical power for correcting a patient's refractive error. As shown schematically in FIG. 1E, a lenticule without such a Fresnel pattern with a similar diameter and optical power will be much thicker, thereby causing a shape transfer effect. Referring to FIG. 1D, while in some cases, the lenticule 110 can be configured for correcting presbyopia, in other cases, it can be formed as a cylindrical lens for correcting astigmatism, by way of example.

In some embodiments, a lenticule according to the present teachings can function as a multi-focal lens. For example, a diffractive pattern or a multi-focal Fresnel pattern can be provided on the anterior or the posterior surface of the lenticule so that the lenticule would provide multiple foci.

A lenticule according to various embodiments of the present teachings, such as the above lenticule 100 can be used as an intrastromal implant in additive ocular surgery.

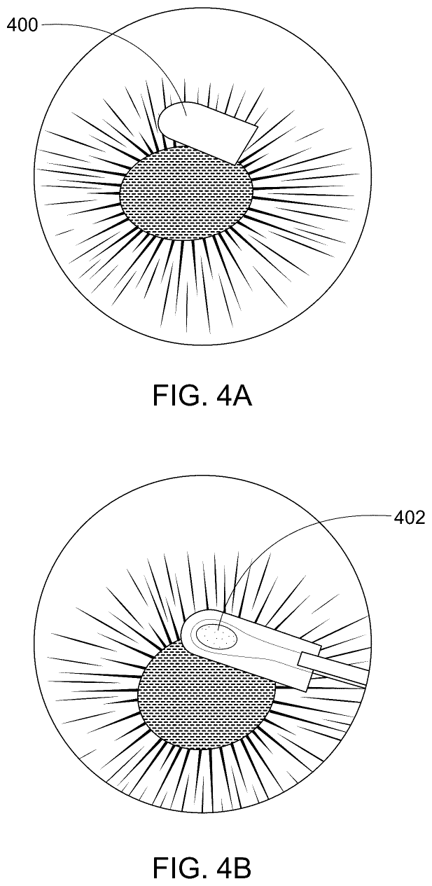

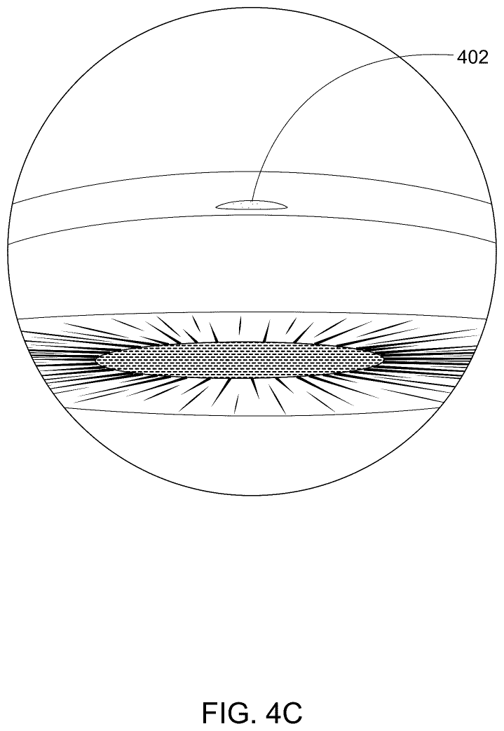

In various embodiments, in use, a lenticule according to the present teachings can be implanted in the stromal layer of a patient's eye to correct a refractive error of the eye. By way of example, with reference to FIGS. 4A, 4B and 4C, the implantation of a lenticule according to the present teachings, e.g., for correcting presbyopia, can be achieved by forming a corneal stromal pocket 400 with a small opening using femtosecond laser pulses (1030 nm) (FIG. 4A). Subsequently, a lenticule 402 according to an embodiment, e.g., for correcting presbyopia, is inserted via the small opening into the pocket formed in the stromal layer (FIG. 4B). In various embodiments, the small thickness of the lenticule, e.g., a thickness less than about 100 microns, allows facile insertion of the lenticule into the pocket formed in the patient's stromal layer.

The lenticule is centered on the patient's visual axis (FIG. 4C). As noted above, in various embodiments, the small size of the lenticule (e.g., a diameter less than about 2 mm) can accommodate some misalignment of the lenticule relative to the visual axis of the patient's eye. In other words, the small size of the lenticule and its ability to correct a refractive error without shape transfer allow a less stringent requirement for the alignment of the lenticule, and more specifically, the alignment of the optical axis of the lenticule, with the visual axis of the recipient's eye.

### Method of Fabrication

In one method according to an embodiment, a lenticule according to the present teachings can be fabricated by first cutting a disc-shaped tissue segment from donor stroma, preferably by preserving the Bowman's membrane as the anterior surface. In some embodiments, the diameter of the lenticule is from about 0.5 mm to about 12 mm, from about 3 mm to about 9 mm, from about 4 mm to about 8 mm and from about 5 mm to about 7 mm. The tissue segment can be sliced and/or further shaped or cut in such a manner that the desired shape is obtained during the slicing procedure. By way of example, cutting of the donor stroma can be performed mechanically, e.g., with a microkeratome or the like, e.g., by laser processing, e.g., by photo-cleavage with a femtosecond laser. Cutting may be performed, for example, with instruments such as those disclosed in International Patent Application No. PCT/IB2016/054793, entitled “Surgical Apparatus and Blade Elements for Slicing Lamellar Segments from Biological Tissue,” herein incorporated in its entirety by reference.

Lenticules can also be obtained by femtosecond laser ablation, excimer laser ablation, or by cutting the donor stroma with a water jet. If preservation of the anterior segment is not necessary, lenticules can also be obtained by Small Incision Lenticule Extraction (SMILE) techniques, disclosed for example in U.S. Pat. No. 6,110,166 entitled “Method For Corneal Laser Surgery,” also herein incorporated in its entirety by reference.

In various embodiments, the cut donor stroma is decellularized to produce lenticules with reduced potential for adverse reaction on the part of the patient to immunogens of cellular origin. By way of example, the decellularized lenticules are between 90 percent to 100 percent, or preferably between 95 percent to 99.99 percent, or between 98 and 99.9 percent, free of cells and/or cellular remnants. Only about 2 percent of the typical cornea is composed of cells. The other 98 percent is largely extracellular matrix (ECM), primarily collagen, water, GAGs and proteoglycans. The preferred and mostly practiced method to characterize the amount of cellular remnants after the decellularization process is based on detection of RNA residues. These methods are very sensitive and of high specificity. These measurements are typically normalized to the amount of RNA present in lenticule before the decellularization step. Without reciting every possible sub-range between 90% and 99.99%, it should be clear that all such sub-ranges are contemplated and considered part of the invention.

For example, the lenticules produced can be 90%, 95%, 99% to 99.7%, 99.7% to 99.9%, or better, free of native cellular materials (e.g., as measured by RNA residual content). In other words, the amount of cellular material remaining in the lenticule, as measured by residual RNA content, can be less than one percent, or less than 0.1 percent, or less than 0.01 percent by weight of the original RNA content. In some cases, a significant amount of decellularization can occur by virtue of the lenticule extraction itself. As much as 95 percent of the total corneal cellular content resides in the epithelium and the endothelium, which can be mechanically discarded leaving only corneal stroma for the further decellularization.

The removal of cellular material from the donor stroma (decellularization) can be accomplished using a variety of techniques. In one preferred embodiment, the cellular material of the cornea is removed by chemical treatment. The chemicals used to lyse and remove cells from the cornea can include acids, bases, surfactants (e.g., sodium tetradecyl sulfate (STS)), ionic detergents (e.g., sodium dodecyl sulfate (SDS)), non-ionic detergents (e.g., Triton X-100), and zwitterionic detergents.

Alternatively, or in addition, the cellular material of the cornea can be removed using an enzymatic treatment. Lipases, thermolysin, galactosidases, nucleases, trypsin, endonucleases and exonucleases can be used to remove the cellular material from the cornea. In some embodiments, the cellular material of the cornea is removed using physical techniques. These physical techniques include methods used to lyse, kill, and remove cells from the matrix of a tissue through the use of temperature, pressure, and/or electrical disruption. Temperature-based decellularization methods can include rapid freeze-thaw protocols. Such temperature-based methods conserve the physical structure of the ECM scaffold. Pressure decellularization involves the controlled use of hydrostatic pressure at high temperatures to avoid unmonitored ice crystal formation that could damage the scaffold. Electrical disruption of the plasma membrane is another option to lyse the cellular material in the cornea.

In various embodiments, the lenticule can be further treated to exhibit even lower immunoreactivity due to the degradation of immunogenic epitopes. This is an important step when using xenogeneic donations. For example, two non-human epitopes that may be present in xenograft tissue are N-Glycolylneuraminic acid (Neu5GC) and Galactose alpha-1,3-galactose (Alpha-Gal). These undesirable epitopes are present not only inside or on the surface of stromal cells; a small fraction of the epitopes may be embedded in the glyco-amino-glycans (GAGs), also known as mucopolysaccharides, that wrap around ECM collagen fibrils. In certain embodiments, such epitopes can be removed or conformationally altered (to neutralize the immunogens) by enzymatic treatments, such as kinase or galactosidase treatments, and additional washing. Alternatively, corneal tissue may be harvested from knockout transgenic pigs which lack epitopes, thus producing non immunogenic lenticules without requiring a degradation step. In some instances, it can also be preferable to remove epithelial and/or endothelial cell layers or residues from the lenticule prior to epitope neutralization. This can be accomplished by scraping, e.g., with a scalpel, or by rubbing, e.g., with an abrasive material of suitable roughness. Further teachings regarding decellularization of the corneal donor tissue as well as the degradation of immunogenic epitopes can be found in U.S. Patent Application No. 2021/0113737 entitled “Stabilization of Collagen Scaffold,” which is herein incorporated by reference in its entirety.

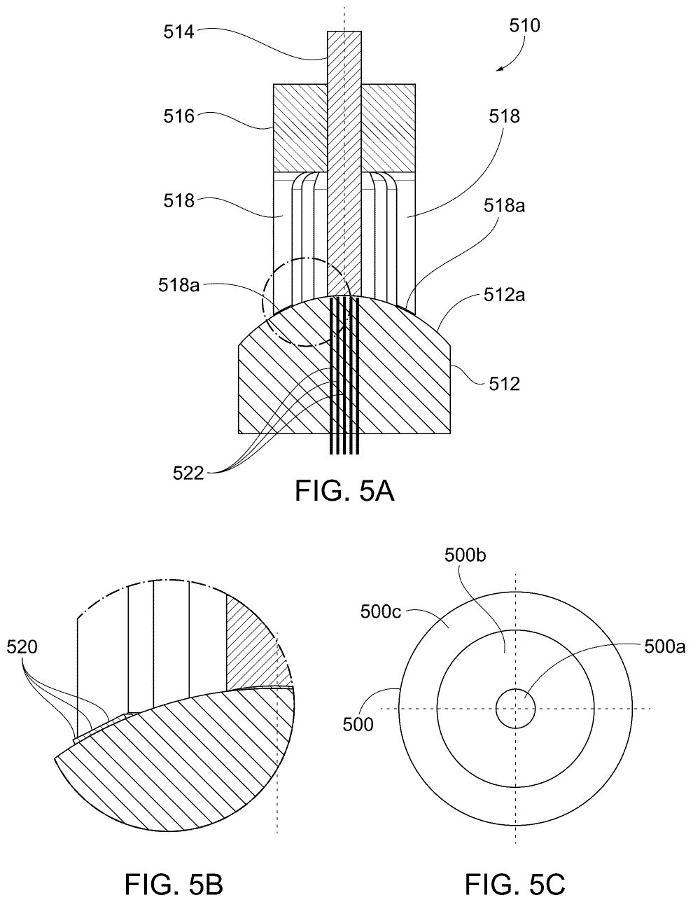

Subsequently, the scaffold can be subjected to controlled compression and stretching followed by exposure to UV radiation to generate a lenticule according to the present teachings, as discussed in more detail below. For example, FIG. 5C schematically depicts a scaffold 500 that is cut from a donor corneal tissue and has been decellularized and treated to lower its immunoreactivity. The scaffold 500 is cut so as to have a thin inner (central) portion 500a, e.g., with a thickness of about 50 microns, a thinner middle portion 500b, e.g., with a thickness in a range of about 15 microns to about 20 microns, and a thicker outer portion 500c, e.g., with a thickness of about 100 to 200 microns.

With particular reference to FIGS. 5A and 5B, the scaffold 500 can be placed in an apparatus 510 that can apply controlled compressive and stretching forces to the scaffold so as to form a lenticule according to the present teachings. The apparatus 510 includes a substrate 512 having a convex hemispherical upper surface 512a, e.g., with a convex radius of curvature of about 7.8 mm, where the scaffold 500 can be placed over the hemispherical surface 512a. For example, the substrate 512 can be formed of a material, such as fused silica, that is transparent to ultraviolet (UV) radiation that can be utilized to cause crosslinking of the collagen fibers within the scaffold, as discussed in more detail below.

The apparatus 510 further includes a central post 514 that is supported by a collar 516 and can be moved along an axial direction (e.g., via a motor not shown in the figure) to apply compressive pressure to the central portion 500a of the scaffold 500. Means for moving the post along the axial direction are not shown as they include well known structures. More specifically, a lower surface 514a of the post 514 has a concave hemispherical shape with a radius of curvature having a value that is somewhat different from the value of the radius of curvature of the hemispherical surface 512a of the substrate 512. By way of example, the lower surface 514a of the post 514 can have a radius of curvature about 4.3 mm.

The lower hemispherical surface 514a of the post 514 is positioned above the central portion 500a of the scaffold 500 and can be pressed down to apply a compressive force to the central portion 500a of the scaffold 500. In particular, the compressive force applied to the central portion of the scaffold is generally perpendicular to the lamellar structure of the collagen fibers within the scaffold so as to preserve the lamellar structure of the collagen fibers as the scaffold is compressed.

The apparatus 510 further includes a plurality of fingers 518 that extend down from the collar 518 and are distributed symmetrically about the post 514. By way of example, the apparatus can include 32 such fingers. The bottom end 518a of each of the fingers 518 has a curved shape that conforms to the hemispherical shape of the upper surface 512a of the hemispherical substrate 512 and is positioned over the thicker outer portion of the scaffold to apply a stretching force to the scaffold.

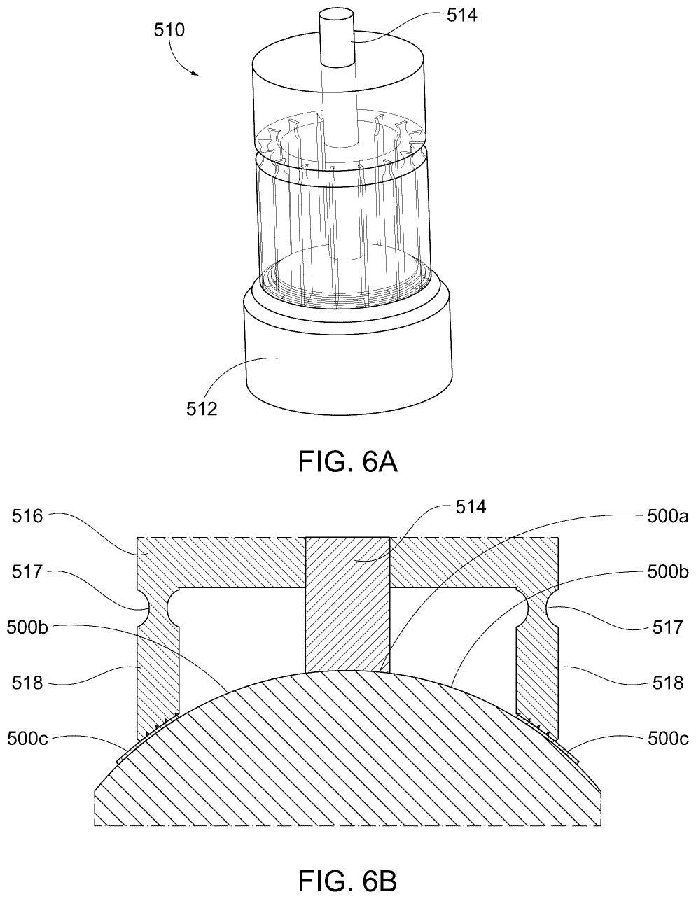

More specifically and with particular reference to FIG. 5B, each of the fingers 518 includes a plurality of tissue-engaging members 520, e.g., in the form of a plurality of miniature, but sharp concentric surface undulations, which grip the outer portion of the scaffold during the stretching of the scaffold. As shown in FIG. 6B, in some implementations of the apparatus, each of the fingers 518 can include a hinge portion 517 that can allow the remainder of the finger to flex in response to the application of a downward force to the finger, thereby facilitating the stretching of the lenticule, as discussed in more detail below. It is noted that the hinge portion 517 is not visible in FIG. 5A.

In use, as the post 514 is pressed down onto the central portion 500a of the scaffold, the fingers 518 are also pressed down, e.g., via application of a downward force to the collar 516, such that the flexing of the hinge portions 517 facilitates a radial motion of the fingers 518, which apply a stretching force via the tissue-engaging members 520 to the outer thicker portion 500c of the scaffold. It is noted that means for moving and controlling the motion of the post and the fingers, such as vertical guiding/sliding/restraining shafts and associated components, are not shown in the figures for the sake of simplicity as such means are readily known to those having ordinary skill in the art.

The stretching force is transmitted via the thin middle portion of the scaffold to its central portion. The transition in the thickness of the scaffold from the outer thicker portion to the middle thinner portion can smooth out potential variations in the strength of the stretching force applied to the central portion of the scaffold, thereby resulting in application of a substantially homogeneous stretching force to the central portion of the scaffold, which will form the intracorneal lenticule.

In various embodiments, the pressing-stretching process is paused intermittently, e.g., for a few minutes, during the fabrication process, e.g., periodically, and then resumed again. It has been discovered that such pausing of the pressing-stretching process allows for better stretching of the scaffold as it can lower the tangential frictional coefficient.

After sufficient compactification of the central portion of the scaffold is achieved, while the scaffold is maintained in a vertically compressed and radially stretched state, the scaffold (and more specifically the central portion of the scaffold) is exposed to UV radiation to cause crosslinking of at least a portion of the collagen fibers within the central portion of the scaffold. In particular, with reference to FIG. 5A, the central portion of the scaffold can be exposed to UV radiation rays 522 through the UV-transparent substrate to cause the crosslinking of the collagen fibers. By way of example, the wavelength of the UV radiation can be in a range of about 220 nm to about 360 nm and the fluence of the UV radiation can be in a range of about 15 J/cm² to about 2500 J/cm².

After completion of the crosslinking step, the scaffold can be removed from the device and the central portion of the scaffold is excised (trephinated) to be used as a lenticule in a manner disclosed herein.

In various embodiments, a lenticule according to the present teachings can provide several advantages. For example, the small thickness of such a lenticule allows its facile implantation into the recipient's cornea. Further, the greater refractive index of the lenticule relative to the recipient's stroma allows correcting various refractive errors without imparting a shape change to the anterior surface of the recipient's cornea.

By way of example, such characteristics coupled with a small diameter of a lenticule allow correcting a patient's near vision while almost entirely preserving the patient's distant vision performance. For example, in laboratory tests, on average, an observed reduction in the patient's distant vision performance was no more than one line of Snellen diagram.

Without being limited to any particular theory, in various embodiments, the reduction or complete elimination of shape transfer minimizes, or even prevents, the influence of a transitional zone on the distance vision, which can occur due to shape transfer. This feature (i.e., the lack of shape transfer) significantly reduces, and in some embodiments substantially eliminates, the sensitivity of the lenticule to small positional and/or lateral placement errors relative to the visual axis, such as those that may occur during ophthalmic surgical procedures (such placement errors are common in ophthalmic surgery theaters).

This insensitivity to misplacement errors can also allow for bilateral usage of the lenticule, which is difficult to achieve by current presbyopic shape transfer lenticule technology, and consequently it is not being practiced.

It is noted that generally the stromal depth at which a lenticule is placed (i.e., the depth of a stromal pocket in which a lenticule is placed) can influence the optical power as well as the diameter of the main and transitional zones induced by shape transfer. More specifically, the deeper the lenticule's placement, the smaller/weaker is the shape transfer effect (and hence the smaller is the shape transfer-induced optical power). In various embodiments, a lenticule according to the present teachings can be implanted in a subject's stroma at a depth of 330 micrometer (microns) or more, relative to the anterior surface of the corneal epithelium).

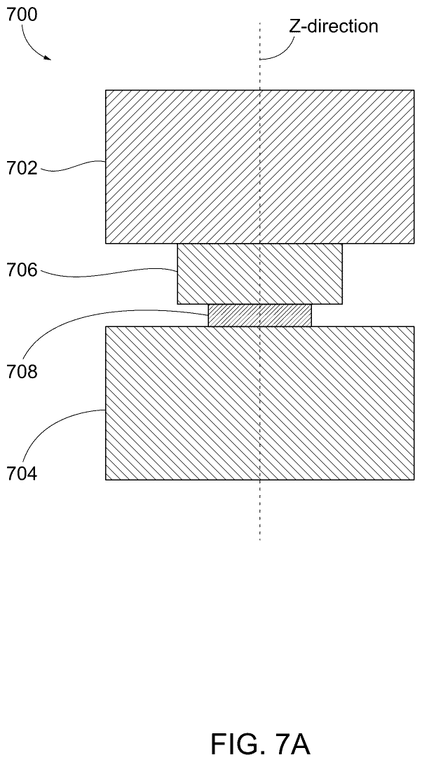

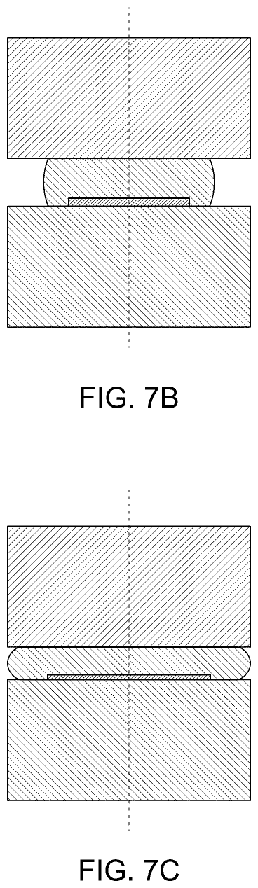

With reference to FIGS. 7A, 7B, and 7C, an apparatus 700 according to an embodiment for exerting controlled compression and stretching forces on a decellularized lenticule includes a first stamping component 702 (herein also referred to as the top stamping component 702) and a second stamping component 704 (herein also referred to as the bottom stamping component 704), which are positioned in a spaced-apart relationship along a vertical direction (herein also referred to as the Z-direction) to form a gap therebetween such that a decellularized corneal scaffold 708 can be positioned in the gap between the two stamping components. A compliant/elastic rubber-like element 706 (e.g., formed of rubber) is affixed to a lower surface of the top stamping component.

In this embodiment, the top stamping component 702 is operatively coupled to a mechanism capable of inducing and controlling changes in the vertical size of the gap between stamping components 702 and 704. A variety of mechanisms can be employed. By way of example, such a mechanism can be a mechanical press, a hydraulic actuator, etc. As shown schematically in FIG. 7B, upon activation of the mechanism, the stamping component 702 moves vertically toward the second stamping component 704 so as to cause the contact of a lower surface of the attached compliant rubber-like element 706 with the decellularized scaffold 708, thereby applying a compressive force along the vertical direction to the decellularized corneal scaffold. This compressive force is generally perpendicular to the lamellar structure of the collagen fibers within the scaffold so as to preserve the lamellar structure of the collagen fibers as the scaffold is compressed. The overall shape of the lenticule is not necessarily flat. A matching curvature similar to human stroma/cornea curvature (e.g., a curvature on the order of R=7.7 [mm]) may be advantageously imprinted.

The compressive force also causes the rubber-like element 706 to stretch in a horizontal direction, i.e., in a direction perpendicular to the direction of the vertical compressive force. This lateral expansion of the compliant rubber-like element 706 results in the exertion of a tensile force in a plane perpendicular to the vertical direction on the decellularized scaffold. In this manner, the decellularized scaffold is subjected to both a vertical compressive force and a horizontal/lateral tensile force. Through the application of the combined compressive and tensile forces in a manner discussed above, an anisotropy in the spatial distribution of the collagen fibers along the vertical and horizontal/lateral directions can be maximized while preserving the optical clarity of the corneal scaffold for intrastromal implantation.

The application of the compressive and tensile forces continues until a desired thickness and lateral stretch of the decellularized corneal scaffold is achieved, as shown schematically in FIG. 7C. Subsequently, the decellularized scaffold can be subjected to UV radiation, preferably while the lenticular body remains under compression, to cause crosslinking of at least a portion of the collagen fibers within the scaffold, thereby structurally solidifying a lenticule exhibiting birefringence and being suitable for intrastromal implantation. As discussed above, such crosslinking of the collagen fibers can strengthen the mechanical stability of the scaffold and inhibit its swelling when implanted in a patient's stroma. In some embodiments, the lower stamping member 704 can be formed of a UV transparent material to allow irradiation of the decellularized corneal scaffold while it remains under the compressive and tensile forces. In various embodiments, the relative lateral expansion of the lenticule is less than about 50%. For clarity of explanation, in FIG. 7C, the radial expansion of the lenticule is exaggerated beyond an expansion that generally occurs in various embodiments.

### EXAMPLE

A presbyopic lenticule was fabricated from corneal tissue extracted from porcine donor based on the above teachings. The lenticule had a disk-like shape with convex anterior and posterior surfaces and a diameter of 1.9 mm.

The refractive index of the lenticule along its optical axis was measured by evaluating the lenticule's focal length/distance when the lenticule was immersed in the following fluids in this order (the refractive index of each fluid is provided in parenthesis after the fluid's name): water (n_{water}=1.3330), Nonan (n_{Nonan}=1.409), Decan (n_{Decan}=1.412), UnoDecan (n_{UnoDecan}=1.418), TetraDecan (n_{TetraDecan}=1.4278). Nonan, Decan, UnoDecan and TetraDecan are immiscible in water.

The optical power P of the lenticule when immersed in a fluid of known refractive index nF is provided by the following relation:

$$P = ( nL - nF ) / Reff$$

where,
nL denotes the lenticule's refractive index along the lenticule's optical axis to be determined and Reff denotes the effective radius of curvature of the lenticule. Here is assumed that nL>nF. The following equation provides the relation between Reff and Rconvex and Rconcave:

$$Reff = 1 / ( 1 / Rconvex - 1 / Rconcave )$$

where,
Reff is the second unknown to be determined. Accordingly, measurements using at least two different fluids are required to arrive at the refractive index of the lenticule along its optical axis.

Once the lenticule's optical power P is determined, the lenticule's focal length in any medium, inclusive of air, can be determined.

The measurement data of the focal lengths of the lenticule in the above fluids together with a square minimization technique was used to derive a value of 1.49 for the refractive index of the lenticule along its optical axis (i.e., nL=1.49).

The refractive index of the lenticule along a tangential direction was not measured. However, the following procedure can be utilized to measure the refractive index of the lenticule along a tangential direction using a refractometer. Specifically, the lenticule's concave side can be placed on the refractometer's plate. The separation between the refractometer's flat surface and the lenticule's concavity can be eliminated by exerting a small distortive force to the lenticule to effectively flatten its concave side. The refractometer can then be used to measure the tangential refractive index of the lenticule. Due to its small size, it is expected that the flattening of the lenticule's concave side not to introduce any significant error in the measurement of its tangential refractive index. It is also feasible to measure a small lenticule's tangential refractive index by trephination of a small central portion of the lenticule, e.g., a portion with a surface area of about 1 mm². But such a technique leads to the destruction of the lenticule.

Those having ordinary skill in the art will appreciate that various changes can be made to the above embodiments without departing from the scope of the invention.

1. A collagenous lenticule for use in intrastromal implantation, comprising:
a decellularized lenticular body derived from a corneal donor source having an anterior surface and a posterior surface;
said decellularized lenticular body comprising collagen fibers distributed therein such that the lenticular body exhibits birefringence to visible light and further exhibits sufficient optical clarity for intrastromal implantation.

2. The collagenous lenticule of claim 1, wherein said lenticular body exhibits a greater refractive index for visible light along an optical axis of the lenticular body relative to at least one axis perpendicular to the optical axis.

3. The collagenous lenticule of claim 1, wherein said birefringence of the lenticular body is characterized by a difference in a range of about 0.005 to about 0.1 for a refractive index of the lenticular body along at least two different spatial orientations within the lenticular body.

4. The collagenous lenticule of claim 3, wherein said at least two orientations comprise a perpendicular orientation and a tangential orientation.

5. The collagenous lenticule of claim 4, wherein said perpendicular orientation is along an optical axis of the lenticular body and said tangential orientation is along a direction orthogonal to the optical axis.

6. The collagenous lenticule of claim 5, wherein the optical axis of the lenticular body coincides approximately with an axis of symmetry of the donor's cornea.

7. The collagenous lenticule of claim 6, wherein the optical axis of the lenticular body is configured to be positioned along a visual axis of a recipient eye receiving the lenticule.

8. The collagenous lenticule of claim 4, wherein said perpendicular orientation extends along an axis that is orthogonal to a putative surface that is tangential to any point on said anterior or said posterior surface and passes through said point and said tangential orientation extends along an axis within said putative surface that passes through said point.

9. The collagenous lenticule of claim 8, wherein said tangential orientation is selected from among axes within said putative surface that pass through said point so as to maximize a difference in the refractive index of the lenticular body between the perpendicular and the tangential orientations.

10. The collagenous lenticule of claim 4, wherein said perpendicular orientation extends along an axis that is orthogonal to a putative surface that is tangential to any point on said anterior or said posterior surface and said tangential orientation extends along an axis within said putative surface that passes through said point.

11. The collagenous lenticule of claim 3, wherein the refractive index of the lenticular body along said perpendicular orientation is greater than an average refractive index of human stromal layer by a value in a range of about 0.01 to about 0.07, wherein optionally said at least one of said perpendicular orientation is along an optical axis of the lenticular body.

12. The collagenous lenticule of claim 11, wherein the refractive index of the lenticular body along said perpendicular orientation is greater than 1.43 by a value in a range of about 0.01 to about 0.07.

13. The collagenous lenticule of claim 3, wherein the refractive index of the lenticular body along said tangential orientation is greater than 1.38, wherein optionally said tangential orientation is orthogonal to an optical axis of the lenticular body.

14. The collagenous lenticule of claim 1, wherein said lenticular body has a maximum lateral dimension of about 12 mm.

15. The collagenous lenticule of claim 14, wherein said lenticular body has a maximum lateral dimension in a range of about 0.5 mm to about 12 mm.

16. The collagenous lenticule of claim 1, wherein said lenticular body has a substantially disk-like shape with a diameter in a range of about 0.5 mm to about 12 mm.

17. The collagenous lenticule of claim 1, wherein said lenticular body has a thickness less than about 100 microns, and optionally in a range of about 10 microns to about 50 microns.

18. The collagenous lenticule of claim 1, wherein a concentration of the collagen fibers in the lenticular body is at least 20%, and optionally at least 25% or at least 30% or at least 40%, or at least 50%, or at least 60%.

19. The collagenous lenticule of claim 1, wherein said collagen fibers are at least partially crosslinked independent of locations of the collagen fibers within the lenticular body.

20. The collagenous lenticule of claim 1, wherein the collagen fibers in the lenticular body substantially retain a layered arrangement of the collagen fibers of the corneal donor source.

21. The collagenous lenticule of claim 1, wherein any remaining cellular material in the decellularized lenticular body, as measured by residual RNA content, is less than one percent, or less than 0.1 percent, or less than 0.01 percent by weight of the original RNA content.

22. The collagenous lenticule of claim 1, wherein the lenticule exhibits low immunoreactivity due to removal or degradation of immunogenic epitopes.

23. The collagenous lenticule of claim 1, wherein the lenticule exhibits a scattering angle, theta, of less than 4 arcminutes, or less than 2 arcminutes, or less than 1 arcminute.

24. The collagenous lenticule of claim 1, wherein said lenticule exhibits an optical clarity characterized by at least 90 percent of visible photons passing through the lenticule with no scattering events.

25. The collagenous lenticule of claim 1, wherein the lenticule exhibits an optical clarity characterized by a maximum scattering angle of visible photons passing through the lenticule being approximately equal to an angle corresponding to a diffraction limited resolution with an apodization of about 3 mm exhibited by said lenticule in the visible portion of the electromagnetic spectrum.

26. The collagenous lenticule of claim 1, wherein said anterior and posterior surfaces of the lenticular body exhibit a curved profile.

27. The collagenous lenticule of claim 26, wherein a center of curvature of the anterior surface coincides with a center of curvature of the posterior surface.

28. The collagenous lenticule of claim 26, wherein said anterior and posterior surfaces of the lenticular body are substantially hemispherical

29. The collagenous lenticule of claim 28, wherein any of said anterior and said posterior surface exhibits a radius of curvature in a range of about 3 mm to about 9 mm.

30. The collagenous lenticule of claim 1, wherein at least one of said anterior and posterior surfaces includes a diffractive or Fresnel structure.

31. The collagenous lenticule of claim 30, wherein said diffractive structure comprises a monofocal Fresnel lens.

32. The collagenous lenticule of claim 30, wherein said lenticule is a multi-focal lenticule.

33-51. (canceled)
